(19) United States
(12) Patent Application Publication
(10) Pub. No.: US 2020/0246531 A1
(43) Pub. Date: Aug. 6, 2020

(54) SYSTEMS AND METHODS FOR DIVERTING BLOOD FLOW IN BLOOD VESSELS

(71) Applicant: Contract Medical International GmbH, Dresden (DE)

(72) Inventors: Tim LENIHAN, Hradec Kralove (CZ); Marcus WENZEL, Priestewitz (DE)

(73) Assignee: Contract Medical International GmbH, Dresden (DE)

(21) Appl. No.: 16/779,072

(22) Filed: Jan. 31, 2020

Related U.S. Application Data
(60) Provisional application No. 62/801,059, filed on Feb. 4, 2019.

(51) Int. Cl. A61M 1/36; A61M 25/00

(52) U.S. Cl. CPC A61M 1/3659; A61M 25/003; A61M 25/007; A61M 2202/0413; A61M 25/0041; A61M 2025/0039; A61M 25/005

(57) ABSTRACT

Devices and methods for perfusing a patient's vasculature are provided. The device preferably includes a cannula with a port and a retractable sheath disposed over the port of the cannula. When the cannula is positioned within the patient's vasculature, the retractable sheath may be retracted to expose the port to divert blood flow towards another portion of the patient's vasculature, for example, to prevent ischemia of the patient's lower extremities. The device may be used during heart surgery, percutaneous heart or circulatory procedures, or other cardiac interventional procedures and may be used in conjunction with another device such as an extracorporeal membrane oxygenation (“ECMO”) machine.

### CROSS-REFERENCE TO RELATED APPLICATIONS

This application claims the benefit of priority of U.S. Provisional Patent Application No. 62/801,059, filed Feb. 4, 2019, the entire contents of which are incorporated herein by reference.

### FIELD OF THE INVENTION

This application relates generally to devices and methods for diverting blood flow within a patient's vasculature for example, to prevent ischemia of the patient's lower extremities during heart surgery, percutaneous heart or circulatory procedures, or other cardiac interventional procedures.

### BACKGROUND

Patients suffering from cardiopulmonary failure whose heart and lungs are unable to provide an adequate amount of gas exchange or perfusion are candidates for cardiopulmonary assistance such as Cardiopulmonary Bypass (“CPB”). CPB is a machine that is surgically connected to the patient's vasculature, e.g., via the femoral artery, to remove blood from the patient's body, artificially remove carbon dioxide and oxygenate red blood cells, and return the oxygenated blood to the patient's body. By performing the function of the patient's heart and lungs, CPB provides the surgeon with a completely still surgical field for complex cardiopulmonary procedures. Extracorporeal membrane oxygenation (“ECMO”), which is largely derived from CPB, provides longer term cardiac and respiratory support. Like CPB, an ECMO machine is connected to the patient to remove blood from the patient's body, artificially remove carbon dioxide and oxygenate red blood cells, and return the oxygenated blood to the patient's body as illustrated in FIG. 1. ECMO is generally used post-CPB or in late stage treatment of patients with profound heart and/or lung failure, and is now also used on patients suffering cardiac arrest.

Typically, during ECMO, a cannula is advanced through the femoral artery having a size sufficient to deliver an adequate amount of oxygenated blood to the patient's heart. During the procedure, the cannula partially or wholly blocks the artery, thereby preventing or reducing blood flow to the patient's lower extremities, which may result in, e.g., ischemia of the foot, for patients requiring longer term cardiopulmonary support. For example, heart pumps such as the Impella® (available from Abiomed®, Danvers, Mass., USA), may have a pump head having a diameter >20 Fr, whereas the main shaft of the device is only 9 Fr in diameter. When the pump head is properly positioned to pump blood to the heart, the 9 Fr shaft remains in the blood vessel toward the lower extremities. However, in order to position the pump head at the target site, the introductory sheath must also have a diameter greater than 20 Fr to accommodate the pump head. Thus, the 9 Fr main shaft of the device is disposed within the >20 Fr introductory sheath, providing ample space between the outer diameter of the 9 Fr shaft and the inner diameter of the >20 Fr sheath, though preventing blood to flow therein.

Attempts have been made to avoid ischemia during such cardiopulmonary procedures including advancing the distal end of the cannula into the aorta, which requires a more invasive and dangerous procedure, or utilizing an additional cannula downstream of the first cannula for delivering oxygenated blood toward the patient's lower extremities as shown in FIG. 2, which requires additional tools, e.g., a cannula having a Y-connector, and an additional entry site into the patient's vasculature. Cannulas have been designed with additional apertures to allow blood to flow both toward the heart and the lower extremities. For example, U.S. Pat. No. 5,171,218 to Fonger is directed to an arterial cannula having a diverting side hole for simultaneously delivering blood to the body and the lower extremities while maintaining adequate flow to the heart. Specifically, the cannula of Fonger includes a sloped barb on its exterior adjacent to the diverting side hole to position the side hole away from the vessel wall.

In addition, U.S. Pat. No. 8,795,253 to Moshinsky is directed to a bi-directional perfusion cannula. Specifically, the cannula has a first aperture at a distal end for blood flow into the artery in the direction of insertion, and a second aperture positioned slightly rearward of an elbow of the cannula for directing blood flow in a second direction opposite the first. U.S. Pat. No. 6,186,981 to Cho is directed to a cavo-atrial cannula having first and second openings for removing blood from the body. Specifically, the second opening of Cho is positioned adjacent to a bend of the cannula. U.S. Pat. No. 9,981,119 to Walther is directed to a bi-directional cannula for perfusing blood in two directions. Specifically, Walther describes a cannula having a substantially tubular extension that moveably protrudes through a second opening positioned at a bend of the cannula. The movement of the tubular extension from within the cannula to outside the cannula is selectively controlled by a control line disposed within the lumen of the cannula.

Accordingly, there exists a need to more efficiently and minimally invasively provide cardiopulmonary support by diverting blood flow in the patient's vasculature.

### SUMMARY OF THE INVENTION

The present invention overcomes the drawbacks of previously-known devices by providing devices and methods for diverting blood flow in a patient's vasculature. In accordance with one aspect, the device includes a cannula having proximal region, a distal region having an outlet, and a lumen extending there between. The outlet is sized and shaped to be in fluid communication with a blood vessel, e.g., the Superficial Femoral Artery. The lumen of the cannula may be sized and shaped to receive a guidewire and optionally a dilator. In addition, the device includes a retractable sheath disposed on the cannula, the retractable sheath transitionable between a first delivery position and a second deployed position, wherein, when the retractable sheath is in the second deployed position, blood flowing through the cannula is permitted to flow in an antegrade direction, e.g., in the direction of natural flow from the heart toward the patient's lower extremities, and a retrograde direction, e.g., against the natural flow of blood, within the blood vessel. The device may include a pump, e.g., an extracorporeal membrane oxygenation (ECMO) system, coupled to the proximal region of the cannula for pumping oxygenated blood through the lumen of the cannula. Additionally, the device may include a pull cord coupled to the retractable sheath for transitioning the retractable sheath between the first delivery position and the second deployed position.

In accordance with one aspect of the present invention, the cannula has a side port disposed between the proximal region and the distal region and in fluid communication with the lumen, and the retractable sheath has a side opening sized and shaped to align with the side port when the retractable sheath is in the second deployed position. Thus, blood flowing through the cannula is permitted to flow in the retrograde direction via the outlet and the antegrade direction via the side port and side opening within the blood vessel.

The device further may include a collapsible tube having a first end coupled to the cannula and in fluid communication with the side port and a second end opposite to the first end. The tube is transitionable between a collapsed state when the retractable sheath is in the first delivery position, wherein the tube is disposed between the retractable sheath and the cannula, and an expanded state when the retractable sheath is in the second deployed position, wherein the tube extends from the cannula so that the second end forms a second outlet. Thus, blood flowing through the cannula is permitted to flow in the retrograde direction via the outlet and the antegrade direction via the second outlet within the blood vessel. For example, the tube may be formed of a wire mesh encapsulated by a flexible membrane. Moreover, a surface of the side opening of the retractable sheath between an inner surface and an outer surface of the retractable sheath may be sloped to facilitate extension of the collapsible tube through the side opening as the retractable sheath transitions from the first delivery position to the second deployed position.

In accordance with another aspect of the present invention, the device may include one or more expandable clips coupled to the cannula adjacent to the side port. The one or more clips are transitionable between a collapsed state when the retractable sheath is in the first delivery position, wherein the one or more clips are disposed between the retractable sheath and the cannula, and an expanded state when the retractable sheath is in the second deployed position, wherein the one or more clips extend from the cannula to facilitate alignment of the side port within a blood vessel lumen. Thus, blood flowing through the cannula is permitted to flow in the retrograde direction via the outlet and a antegrade direction via the side port and the side opening within the blood vessel.

In accordance with yet another aspect of the present invention, the device may include an anchor coupled to the distal region of the cannula adjacent to the outlet. The anchor has an expandable portion transitionable between a collapsed state when the retractable sheath is in the first delivery position, wherein the expandable portion of the anchor is disposed within the retractable sheath, and an expanded state when the retractable sheath is in the second deployed position, wherein the expandable portion of the anchor extends beyond the retractable sheath to thereby anchor the cannula to a blood vessel. In addition, the expandable portion of the anchor forms a seal between the cannula and an entry site of the cannula into blood vessel.

In accordance with one aspect of the present invention, a method for perfusing a patient's vasculature is provided. The method includes advancing a distal end of a cannula having an outlet within a blood vessel of the patient, and moving a retractable sheath disposed on the cannula from a first delivery position to a second deployed position to thereby permit blood to flow through the cannula and into the blood vessel in a retrograde direction and an antegrade direction. The method further may include advancing a guidewire and a dilator within the blood vessel of the patient, wherein the distal end of the cannula is advanced over the guidewire and the dilator.

In accordance with another aspect of the present invention, moving the retractable sheath from the first delivery position to the second deployed position aligns a side opening of the retractable sheath and a side port of the cannula, such that blood flow through the cannula exits the outlet in the retrograde direction and exits the side port and side opening in the antegrade direction within the blood vessel. Moreover, moving the retractable sheath from the first delivery position to the second deployed position may transition a collapsible tube extending from the cannula and in fluid communication with the side port from a collapsed state to an expanded state forming a second outlet, such that blood flow through the cannula exits the outlet in the retrograde direction and exits the second outlet in the antegrade direction within the blood vessel. In accordance with another aspect of the present invention, moving the retractable sheath from the first delivery position to the second deployed position transitions one or more expandable clips coupled to the cannula adjacent the side port from a collapsed state to an expanded state to thereby facilitate alignment of the side port within the blood vessel.

In accordance with another aspect of the present invention, the distal end of the cannula is advanced through an entry site in the blood vessel such that the outlet is in fluid communication with the blood vessel without the cannula wholly blocking the blood vessel. Accordingly, moving the retractable sheath from the first delivery position to the second deployed position transitions an expandable anchor disposed on the distal end of the catheter from a collapsed state to an expanded state to anchor the cannula to the blood vessel and to form a seal between the cannula and the blood vessel at the entry site, such that blood flow through the cannula exits the outlet into the blood vessel in an antegrade direction and a retrograde direction.

### BRIEF DESCRIPTION OF THE DRAWINGS

FIG. 1A illustrates an ECMO system connected to a patient during a cardiopulmonary procedure.

FIG. 1B illustrated an arterial cannula having a Y-connector for delivering blood to the patient's lower extremities.

FIG. 2 illustrates an exemplary device for diverting blood flow in a patient's vasculature constructed in accordance with the principles of the present invention.

FIGS. 3A-3D illustrate exemplary steps of operating the device of FIG. 2 in accordance with the principles of the present invention.

FIGS. 4A-4D illustrate exemplary steps of using the device of FIG. 2 within the patient's vasculature in accordance with the principles of the present invention.

FIG. 5 is a flow chart of steps in an exemplary method for diverting blood flow within the patient's vasculature in accordance with the principles of the present invention.

FIGS. 6A-6H illustrate exemplary steps of operating an alternative exemplary device for diverting blood flow in a patient's vasculature in accordance with the principles of the present invention.

FIG. 7 is a flow chart of steps in an alternative exemplary method for diverting blood flow within the patient's vasculature in accordance with the principles of the present invention.

FIG. 8 illustrates an exemplary device for anchoring an arterial cannula to a blood vessel to divert blood flow within the patient's vasculature in accordance with the principles of the present invention.

FIGS. 9A-9D illustrate exemplary steps of using the device of FIG. 8 within the patient's vasculature in accordance with the principles of the present invention.

FIG. 10 is a flow chart of steps in yet another exemplary method for diverting blood flow within the patient's vasculature in accordance with the principles of the present invention.

FIGS. 11A and 11B illustrate another exemplary device for anchoring an arterial cannula to a blood vessel to divert blood flow within the patient's vasculature in accordance with the principles of the present invention.

FIG. 12 illustrates yet another exemplary device for anchoring an arterial cannula to a blood vessel to divert blood flow within the patient's vasculature in accordance with the principles of the present invention.

FIG. 13 illustrates another exemplary device for anchoring an arterial cannula to a blood vessel to divert blood flow within the patient's vasculature in accordance with the principles of the present invention.

### DETAILED DESCRIPTION

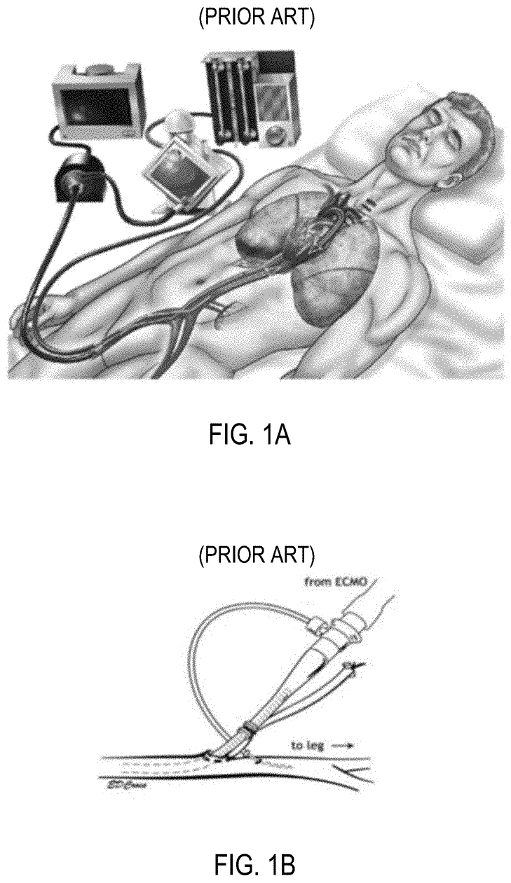

Embodiments of the present invention are directed to devices and methods for diverting blood flow within the patient's vasculature. For example, the device may be used with an extracorporeal membrane oxygenation (“ECMO”) system to divert blood flow toward the patient's lower extremities as oxygenated blood is simultaneously delivered to the patient's heart, to thereby prevent ischemia of the lower extremities during long term cardiopulmonary procedures. As described above, FIG. 1A illustrates an ECMO system connected to a patient during a cardiopulmonary procedure to deliver oxygenated blood to the patient's heart. FIG. 1B illustrates a prior attempt to divert blood flow toward the patient's lower extremities whereby an arterial cannula having a Y-connector is advanced in the patient's vasculature such that one end of the cannula delivers blood toward the heart, and an additional tube requiring an additional entry site into the patient's vasculature delivers blood toward the lower extremities.

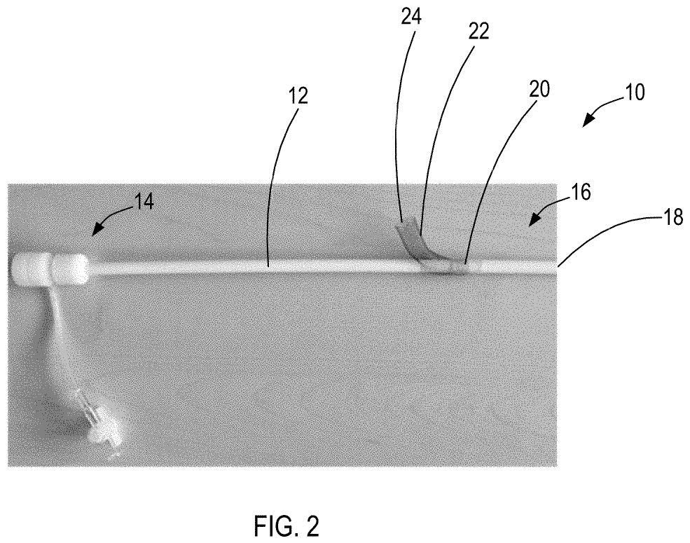

Referring now to FIG. 2, exemplary device 10 for diverting blood flow within a patient's vasculature is provided. Device 10 includes cannula 12 having proximal region 14 and distal region 16 and at least one lumen extending therebetween. Proximal region 14 may be coupled to a pump external to the patient's body for delivering oxygenated blood through the lumen of cannula 12. For example, the pump may be part of an ECMO system.

Cannula 12 has first outlet 18 at the distal end of distal region 16 in fluid communication with the lumen of cannula 12. Distal region 16 of cannula 12 is sized and shaped to fit within the patient's target blood vessel, e.g., the Superficial Femoral Artery, and the lumen is sufficiently sized to deliver an adequate amount of oxygenated blood via first outlet 18 toward the patient's heart during operation of device 10, e.g., during a cardiopulmonary procedure. In addition, cannula 12 has side port 20 positioned along cannula 12 between proximal region 14 and distal region 16. Side port 20 is in fluid communication with the lumen of cannula 12 and is sized to permit an adequate amount of blood to flow therethrough toward the patient's lower extremities.

Device 10 further includes collapsible tube 22 having a first end coupled to side port 20, and a second end forming second outlet 24 in fluid communication with the lumen of cannula 12. Device 10 also has a retractable sheath disposed on cannula 12, though not shown in FIG. 2 to illustrate the connection of tube 22 with side port 20, which will be described in further detail below. Tube 22 may be made of, for example, a wire mesh encapsulated by a flexible membrane to provide suitable flow therethrough. Tube 22 is transitionable between a collapsed state, wherein tube 22 is disposed against the outer surface of cannula 12 between the retractable sheath and cannula 12, and an expanded state, wherein tube 22 extends from side port 20 of cannula 12, such that when blood is permitted to flow through cannula 12, blood exiting first outlet 18 flows in an retrograde direction and blood exiting second outlet 24 flows in a antegrade direction within the patient's vasculature.

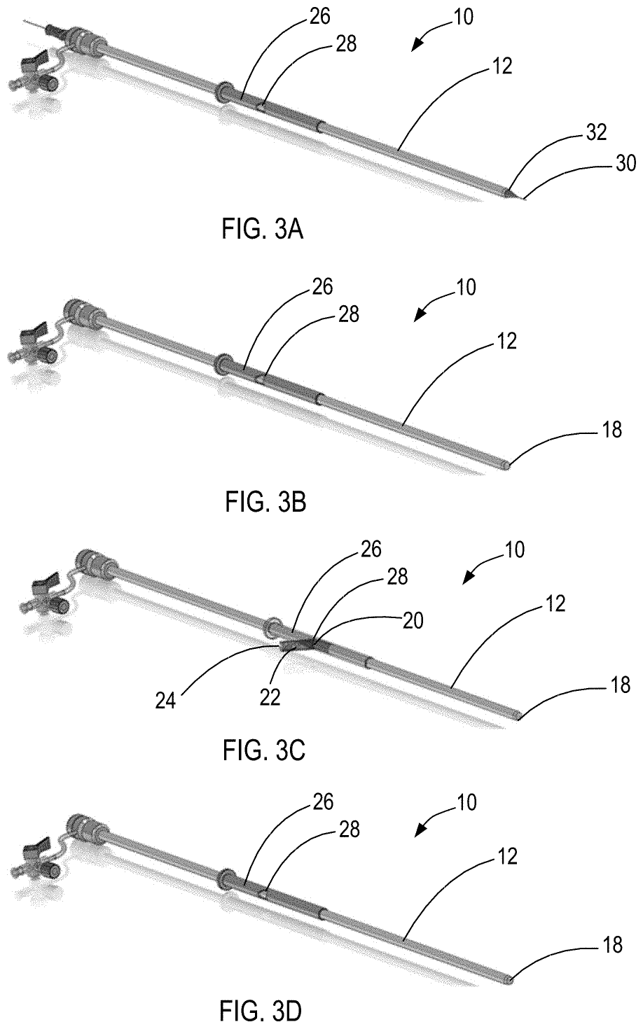

Referring now to FIGS. 3A through 3D, exemplary steps of operating device 10 in accordance with the principles of the present invention are described. As illustrated in FIGS. 3A-3D, device 10 may include retractable sheath 26 disposed on at least a portion of cannula 12. Retractable sheath 26 has side opening 28 sized and shaped to align with side port 20 of cannula 12 to permit tube 22 to extend therethrough. As shown in FIG. 3A, retractable sheath 26 is disposed on cannula 12 in a first delivery position, whereby tube 22 is in the collapsed state between the outer surface of cannula 12 and the inner surface of retractable sheath 26. Accordingly, during delivery of distal region 16 of cannula 12 within the target blood vessel, e.g., the Superficial Femoral Artery, tube 22 is not exposed and side port 20 may be blocked by retractable sheath 26. Cannula 12 may be delivered to the target location within the patient's vasculature over guidewire 30 and optionally dilator 32 as shown in FIG. 3A. Therefore, the lumen of cannula 12 may be sized and shaped to permit guidewire 30 and/or dilator 32 therethrough. FIG. 3B illustrates device 10 after the dilator and guidewire have been removed.

Retractable sheath 26 may then be moved from the first delivery position to a second deployed position whereby tube 22 extends from side port 20 of cannula 12 through side opening 28 of retractable sheath 26 in the expanded state, as shown in FIG. 3C. As retractable sheath 26 moves from the first position to the second position, second outlet 24 of tube 22 is exposed through side opening 28 of retractable sheath 26 and tube 22 self-expands to the expanded state. In the second position, side opening 28 of retractable sheath 26 is aligned with side port 20 of cannula 12 such that tube 22 may extend from side port 20 through side opening 28 to permit blood to flow therethrough. Retractable sheath 26 may move relative to cannula 12 such that either cannula 12 remains in place relative to the patient's vasculature as retractable sheath 26 is moved, or retractable sheath 26 remains in place relative to the patient's vasculature as cannula 12 is moved. For example, device 10 may include an actuator such as a pull cord coupled to retractable sheath 26 for actuating movement of retractable sheath 26.

As illustrated in FIG. 3D, when the cardiopulmonary procedure is completed, or when there is no longer a need to divert blood toward the patient's lower extremities, retractable sheath 26 may be moved from the second deployed position back to the first delivery position to transition tube 22 back to the collapsed state between retractable sheath 26 and cannula 12, thereby preventing blood flow outside port 20 and through tube 22. Device 10 may then be removed from the patient's vasculature.

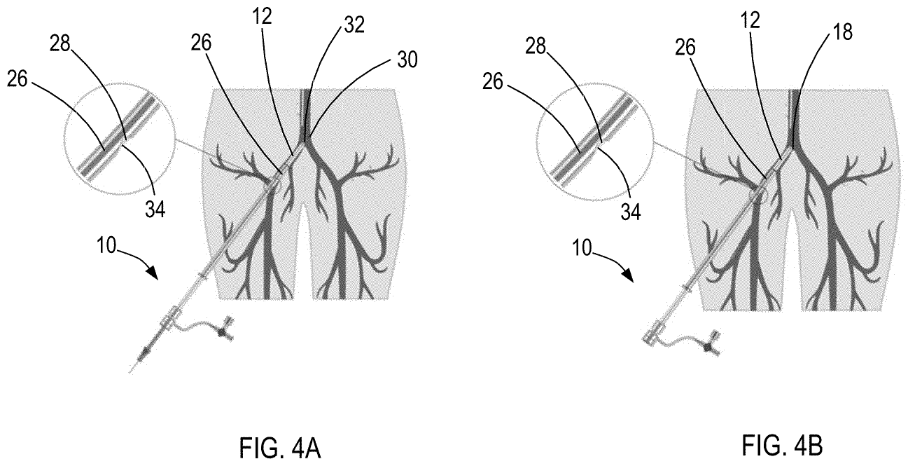

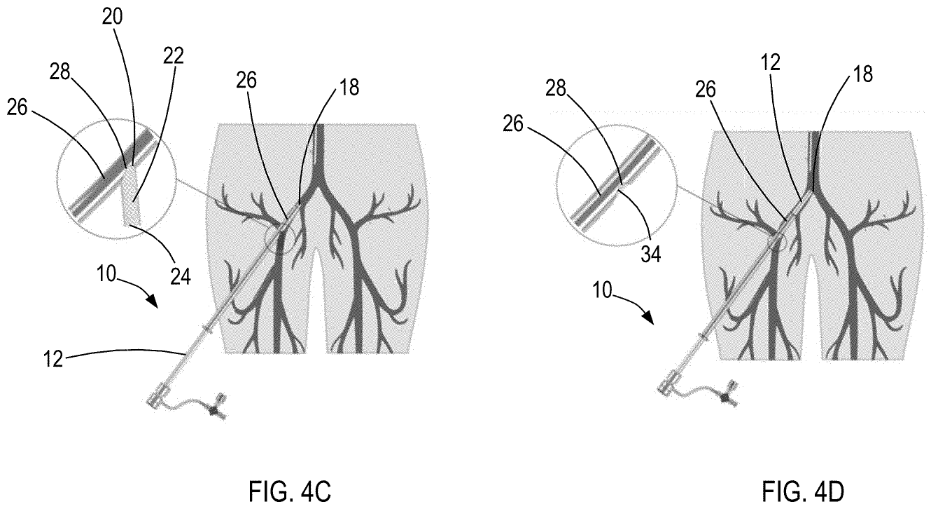

FIGS. 4A through 4D illustrate exemplary steps of using device 10 within the patient's vasculature. As illustrated in FIGS. 4A-4D, the surface of proximal portion 34 of side opening 28 of retractable sheath 26 between the inner surface and the outer surface of retractable sheath 26 is sloped to facilitate extension of tube 22 through side opening 28 as retractable sheath 26 transitions from the first delivery position to the second deployed position. For example, the surface of proximal portion 34 may be angled between 10 and 80 degrees, or preferably between 30 and 60 degrees, relative to the longitudinal axis of retractable sheath 26. Accordingly, as retractable sheath 26 transitions from the first position to the second position, tube 22 engages the sloped surface of proximal portion 34, which guides tube 22 through side opening 28 of retractable sheath 26. Similarly, as retractable sheath transitions from the second position to the first position, the sloped surface of proximal portion 34 guides tube 22 back into the collapsed state between retractable sheath 26 and cannula 12.

FIG. 4A illustrates cannula 12 having retractable sheath 26 disposed thereon in the first delivery position, and dilator 32 positioned within its lumen, advanced over guidewire 30. For example, guidewire 30 may be first delivered to the target site within the patient's vasculature. Dilator 32 may be disposed within the lumen of cannula 12, thereby providing sufficient stiffness to cannula 12 for delivery over guidewire 30 to the target site. As shown in FIG. 4B, guidewire 30 and dilator 32 may then be removed, leaving cannula 12 in the desired location within the target blood vessel, e.g., the Superficial Femoral Artery. As further shown in FIG. 4B, side opening 28 of retractable sheath 26 is aligned with the blood vessel lumen such that tube 22 may be deployed so that second outlet 24 of tube 22 is disposed within the blood vessel upon retraction of retractable sheath 26.

As illustrated in FIG. 4C, cannula 12 may be retracted proximally such that retractable sheath 26 moves distally relative to cannula 12, thereby causing tube 22 to be exposed and extend through side opening 28 of retractable sheath 26 and to transition from the collapsed state to the expanded state within the blood vessel. Accordingly, during operation of device 10, e.g., via an ECMO system, blood is permitted to flow through cannula 12 and exit first outlet 18 in an retrograde direction toward the patient's heart and exit second outlet 24 via side port 20 and side opening 28 in an antegrade direction toward the patient's lower extremities.

Upon completion of the cardiopulmonary procedure, or when diversion of blood flow to the patient's lower extremities are no longer needed, cannula 12 may be moved distally such that retractable sheath 26 moves proximally relative to cannula 12, thereby transitioning tube 22 from the expanded deployed state back to the collapsed state between retractable sheath 26 and cannula 12, as shown in FIG. 4D. Device 10 may then be removed from the patient's vasculature. As will be understood by a person of ordinary skill in the art, the operator may select the amount of retractable sheath 26 that is disposed over and blocking side port 20, e.g., partial expansion of tube 22, and accordingly, selectively regulate and control of flow rate and volume of blood diverted to the patient's lower extremities via second outlet 24.

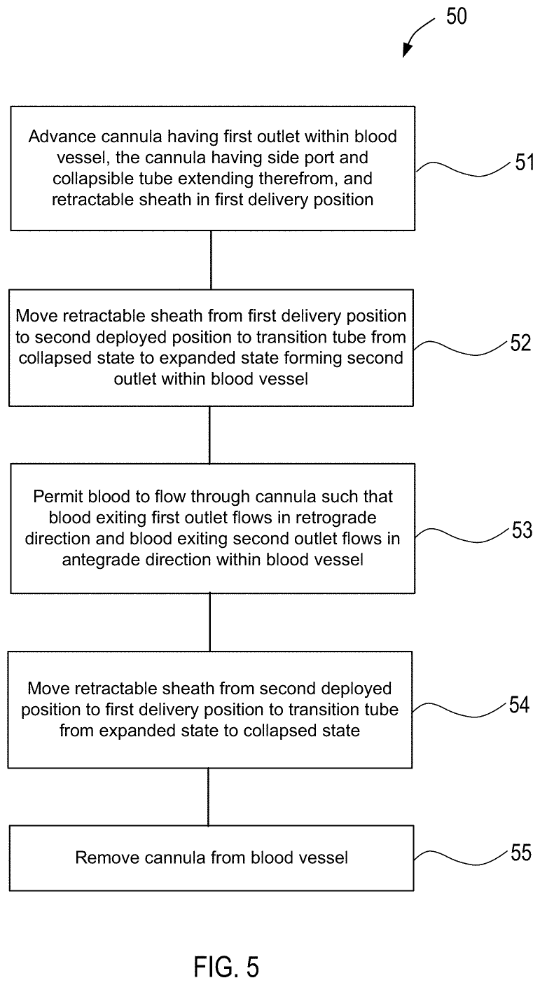

Referring now to FIG. 5, a flow chart of steps in exemplary method 50 for diverting blood flow within the patient's vasculature in accordance with the principles of the present invention is provided. At step 51, distal region 16 of cannula 12 is advanced to the target blood vessel, e.g., the Superficial Femoral Artery, such that first outlet 18 of cannula 12 is disposed within the target blood vessel toward the patient's heart. As described above, a guidewire may be delivered to the target site first, such that cannula 12, having an optional dilator disposed therein, may be advanced to the target site within the blood vessel over the guidewire. Side opening 28 of retractable sheath 26 may be properly aligned with the target blood vessel lumen. As will be understood by a person having ordinary skill in the art, alignment of side opening 28 with the target blood vessel lumen may be verified using visualization methods known in the art such as fluoroscopy.

At step 52, retractable sheath 26 is moved relative to cannula 12 from a first delivery position to a second deployed position, thereby causing tube 22 to extend through side opening 28 and transition from a collapsed state to an expanded state within the blood vessel, such that second outlet 24 of tube 22 is disposed within the target blood vessel toward the patient's lower extremities. At step 53, during operation of device 10, blood is permitted to flow through cannula 12 such that blood exiting first outlet 18 flows in a retrograde direction toward the patient's heart, and blood exiting second outlet 24 flows in an antegrade direction toward the patient's lower extremities. At step 54, retractable sheath 12 is moved relative to cannula 12 from the second deployed position back to the first delivery position, thereby causing tube 22 to transition from the expanded state within the blood vessel to the collapsed state between retractable sheath 26 and cannula 12. At step 55, device 10 may be removed from the patient's vasculature.

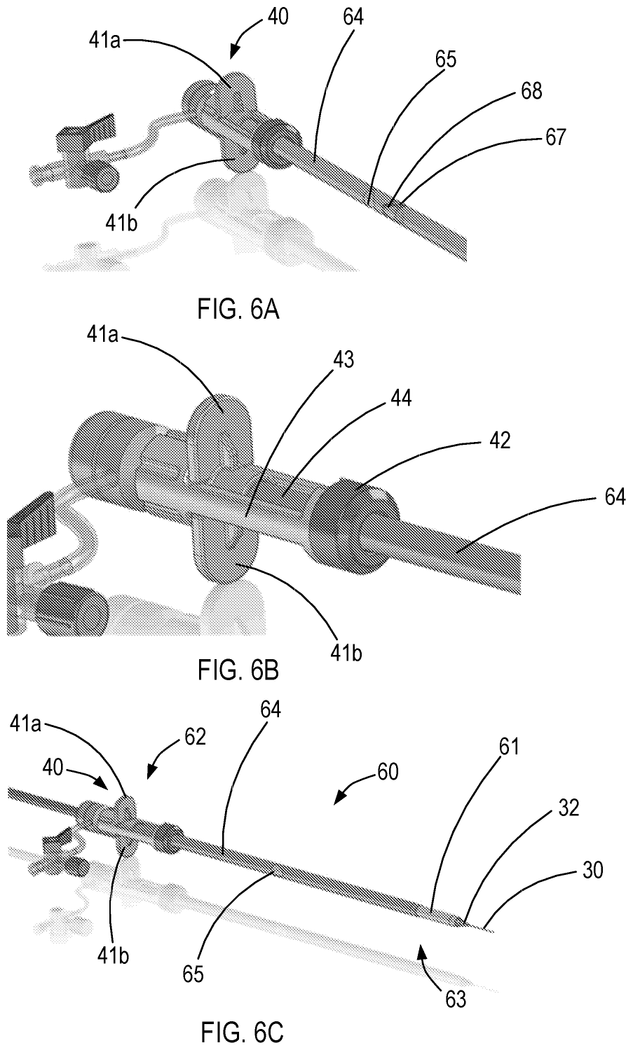

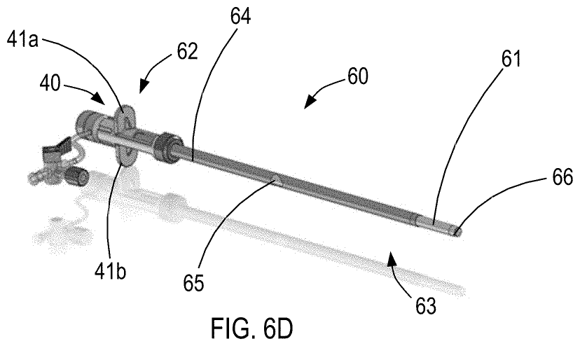

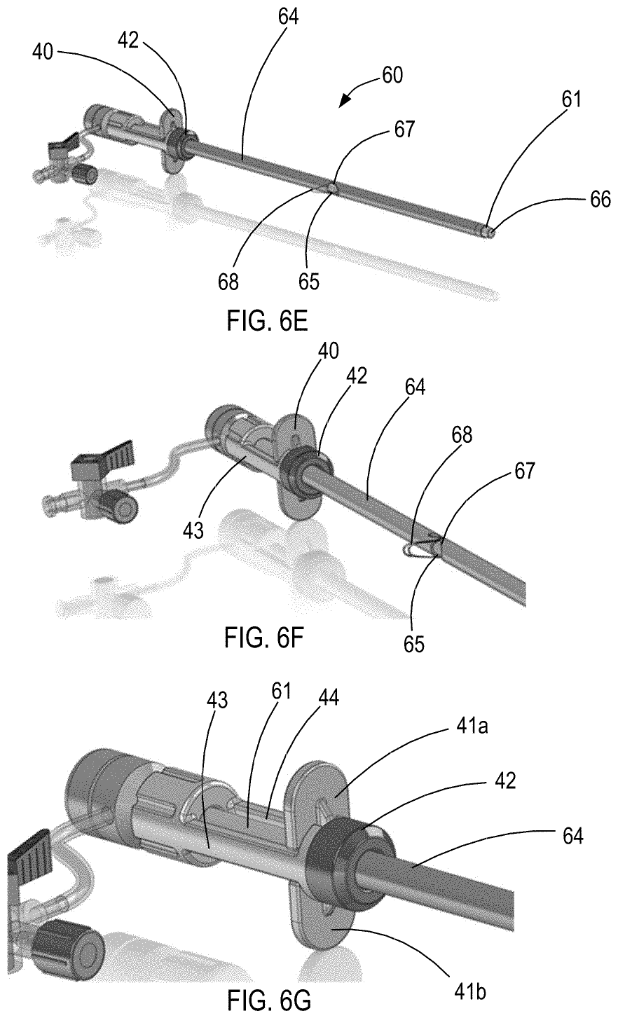

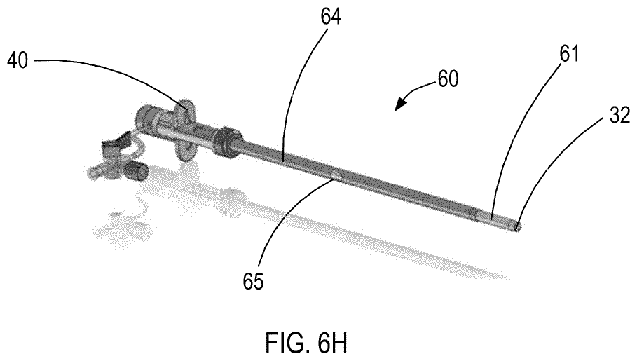

In accordance with another aspect of the present invention, the cannula may include expandable clips for aligning the side port of the cannula within the blood vessel to divert blood flow to the patient's lower extremities during operation. Referring now to FIGS. 6A-6H, exemplary steps of operating device 60 for diverting blood flow within a patient's vasculature is provided. As shown in FIGS. 6A-6H, device 60 includes cannula 61 having proximal region 62 and distal region 63 and at least one lumen extending therebetween. Proximal region 62 may be coupled to a pump external to the patient's body for delivering oxygenated blood through the lumen of cannula 61. For example, the pump may be part of an ECMO system. In addition, proximal region 62 of cannula 61 includes actuator 40 coupled to a proximal end of a retractable sheath disposed on cannula 61 for actuating movement of the retractable sheath relative to cannula 61 as will be described in further detail below.

Cannula 61 has first outlet 66 at the distal end of distal region 63 in fluid communication with the lumen of cannula 61. Distal region 63 of cannula 61 is sized and shaped to fit within the patient's target blood vessel, e.g., the Superficial Femoral Artery, and the lumen is sufficiently sized to deliver an adequate amount of oxygenated blood via first outlet 66 toward the patient's heart during operation of device 60, e.g., during a cardiopulmonary procedure. Moreover, the lumen of cannula 61 may be sized and shaped to receive guidewire 30 and/or dilator 32 therethrough for delivery to the target blood vessel.

Cannula 61 has side port 67 positioned along cannula 61 between proximal region 62 and distal region 63. Side port 67 is in fluid communication with the lumen of cannula 61 and is sized to permit an adequate amount of blood to flow therethrough toward the patient's lower extremities. In addition, device 60 further includes one or more expandable clips 68 coupled to side port 67, and extending outwardly therefrom. Clip 68 may include, for example, one or more expandable wires.

Device 60 further includes retractable sheath 64 disposed on at least a portion of cannula 61. Retractable sheath 64 has side opening 65 sized and shaped to align with side port 67 of cannula 61 to permit clips 68 to extend therethrough, and accordingly to permit fluid flow therethrough. Clips 68 are transitionable between a collapsed state, wherein clips 68 are disposed against the outer surface of cannula 61 between retractable sheath and cannula 61, and an expanded state, wherein clips 68 extend from side port 67 of cannula 61 to align side port 67 and side opening 65 with the lumen of the blood vessel. Accordingly, when blood is permitted to flow through cannula 61, blood exiting first outlet 66 flows in a retrograde direction and blood exiting side port 67 and side opening 65 flows in an antegrade direction within the patient's vasculature.

As shown in FIG. 6A, actuator 40 is in an initial position such that retractable sheath 64 is disposed on cannula 61 in a first delivery position, and clips 68 are in the collapsed state between the outer surface of cannula 61 and the inner surface of retractable sheath 64. Accordingly, during delivery of distal region 63 of cannula 61 within the target blood vessel, e.g., the Superficial Femoral Artery, clips 68 are not exposed and side port 67 may be blocked by retractable sheath 64.

As illustrated in FIG. 6B, actuator 40 may include flaps 41a, 41b extending radially outward for ease of contact with the operator's fingers. In addition, actuator 40 includes frame 43 having opening 44 to permit flaps 41a, 41b, and accordingly retractable sheath 64, to slidably move within opening 44 of frame 43. Opening 44 also prevents rotation of flaps 41a, 41b, thereby permitting retractable sheath to only move along the longitudinal axis of cannula 61, and maintaining side port 67 and side opening 65 in the same longitudinal plane. Frame 43 may also be coupled to stop 42 positioned distal to flaps 41a, 41b, to thereby prevent distal movement of flaps 41a, 41b beyond a desired amount. Accordingly, as flaps 41a, 41b of actuator 40 moves distally within opening 44 of frame 43, retractable sheath 64 is also moved distally relative to cannula 61.

Cannula 61 may be delivered to the target location within the patient's vasculature over guidewire 30 and optionally dilator 32 as shown in FIG. 6C when actuator 40 is in the initial delivery position. For example, guidewire 30 may be delivered to the target blood vessel first, and cannula 61 with optional dilator 32 disposed therein may then be advanced over guidewire 30 to the target blood vessel. FIG. 6D illustrates device 60 after dilator 32 and guidewire 30 have been removed.

Actuator 40 may then be moved from the initial positon to a deployed position to move retractable sheath 64 from the first delivery position to a second deployed position, so that clips 68 extend from side port 67 of cannula 61 through side opening 65 of retractable sheath 64 in the expanded state, as shown in FIGS. 6E-6G. As retractable sheath 64 moves from the first position to the second position, side port 67 of cannula 61 aligns with side opening 65 of retractable sheath 64, and clips 68 self-expand to the expanded state. Specifically, in the expanded state, clips 68 align and maintain side port 67 and side opening 65 with the lumen of the blood vessel. Thus, when blood flow is permitted to flow through the lumen of cannula 61, blood exiting outlet 66 flows in a retrograde direction and blood exiting side port 67 and side opening 65 flows in an antegrade direction toward the patient's lower extremities.

As illustrated in FIG. 6H, when the cardiopulmonary procedure is completed, or when there is no longer a need to divert blood toward the patient's lower extremities, actuator 40 may move retractable sheath 64 from the second deployed position back to the first delivery position to transition clips 68 back to the collapsed state between retractable sheath 64 and cannula 61, thereby preventing blood flow outside side port 67 and side opening 65. Device 60 may then be removed from the patient's vasculature. As will be understood by a person of ordinary skill in the art, actuator 40 may be moved to and maintained in any position between the initial position and the deployed position, thereby allowing the operator to select the amount of retractable sheath 64 that is disposed over and blocking side port 64, e.g., partially open/closed, and accordingly, selectively regulate and control of flow rate and volume of blood diverted to the patient's lower extremities.

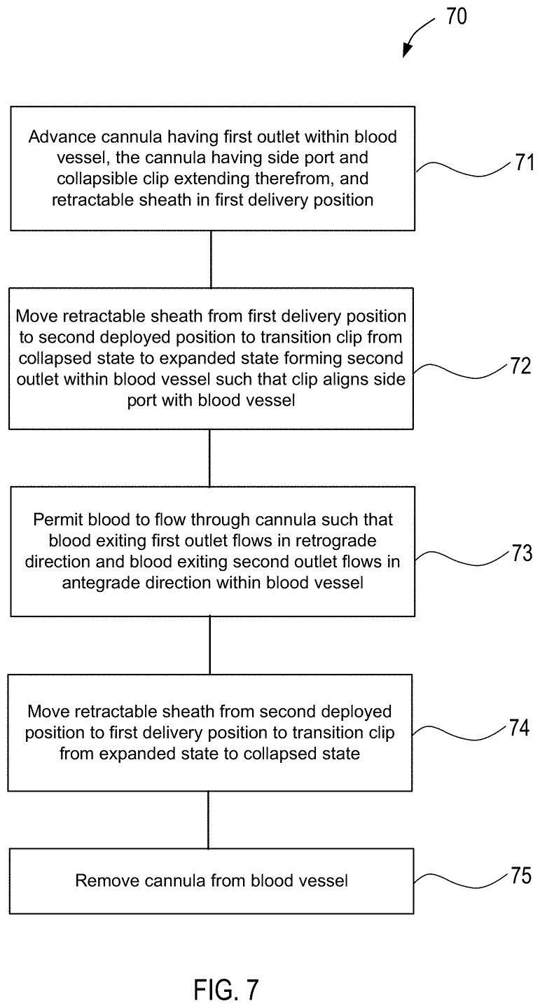

Referring now to FIG. 7, a flow chart of steps in exemplary method 70 for diverting blood flow within the patient's vasculature in accordance with the principles of the present invention is provided. At step 71, distal region 63 of cannula 61 is advanced to the target blood vessel, e.g., the Superficial Femoral Artery, such that first outlet 66 of cannula 61 is disposed within the target blood vessel toward the patient's heart. As described above, a guidewire may be delivered to the target site first, such that cannula 61, having an optional dilator disposed therein, may be advanced to the target site within the blood vessel over the guidewire. Side opening 65 of retractable sheath 64 may be properly aligned with the target blood vessel lumen. As will be understood by a person having ordinary skill in the art, alignment of side opening 65 with the target blood vessel lumen may be verified using visualization methods known in the art such as fluoroscopy.

At step 72, retractable sheath 64 is moved relative to cannula 61 from a first delivery position to a second deployed position, thereby causing clips 68 to extend through side opening 65 and transition from a collapsed state to an expanded state within the blood vessel, such that side port 67 and side opening 65 are aligned with the lumen of the target blood vessel toward the patient's lower extremities. At step 73, during operation of device 60, blood is permitted to flow through cannula 61 such that blood exiting first outlet 66 flows in a retrograde direction toward the patient's heart, and blood exiting side port 67 and side opening 65 flows in an antegrade direction toward the patient's lower extremities. At step 74, retractable sheath 64 is moved relative to cannula 61 from the second deployed position back to the first delivery position, thereby causing clips 68 to transition from the expanded state within the blood vessel to the collapsed state between retractable sheath 64 and cannula 61. At step 75, device 60 may be removed from the patient's vasculature.

In accordance with yet another aspect of the present invention, an anchor may be coupled to the arterial cannula for positioning the outlet of the cannula at a desired position in fluid communication with the lumen of the target blood vessel without blocking the blood vessel. In addition, the anchor functions as a seal and prevents blood from exiting via the entry site of the arterial cannula into the lumen of the blood vessel.

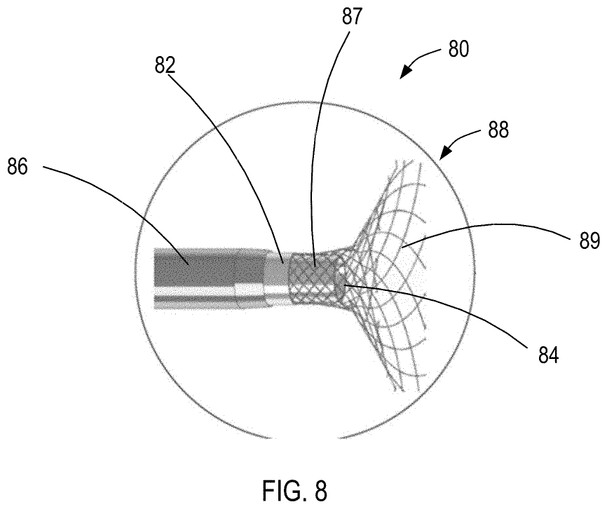

Referring now to FIG. 8, exemplary device 80 for anchoring arterial cannula 82 to a blood vessel to divert blood flow within the patient's vasculature in accordance with the principles of the present invention is provided. Device 80 includes cannula 82 having outlet 84, and expandable anchor 88 coupled to the distal end thereof adjacent to outlet 84. Expandable anchor 88 includes proximal portion 87 which remains fixedly coupled to cannula 82 adjacent outlet 84, and distal expandable portion 89 which is transitionable between a collapsed delivery state and an expanded deployed state. For example, distal expandable portion 89 may be formed of a pre-formed biocompatible metal material. Device 80 further includes retractable sheath 86 disposed on cannula 82. For example, in a delivery state, retractable sheath 86 is disposed over cannula 82 such that both proximal portion 87 and distal expandable portion 89 of anchor 88 are collapsed within retractable sheath 86.

When cannula 82 is advanced to the desired location within the target blood vessel, e.g., via an entry site into the blood vessel, sheath 86 may be retracted to expose distal expandable portion 89 of anchor 88 within the target blood vessel so that distal portion 89 transitions from the collapsed delivery state to an expanded state. In the expanded state, distal portion 89 engages with the vessel wall to anchor cannula 82 to the blood vessel wall such that outlet 84 is in fluid communication with the lumen of the blood vessel in a manner that cannula 82 does not protrude too far into the blood vessel, and thus does not block blood flow within the blood vessel. In addition, anchor 88 may be formed from a wire mesh encapsulated by a flexible membrane such that as distal portion 89 anchors cannula 82 to the blood vessel, distal portion 89 also forms a seal around the cannula adjacent to outlet 84 and the blood vessel to prevent blood from exiting the blood vessel via the entry site. Accordingly, when blood is permitted to flow through the lumen of cannula 82, blood exits outlet 84 and is permitted to flow in both antegrade and retrograde directions within the blood vessel as cannula 82 is not blocking the blood vessel. Retractable sheath 86 may be moved back to the delivery position to transition distal portion 89 of anchor 88 back into sheath 86, so that device 80 may be removed from the patient's vasculature.

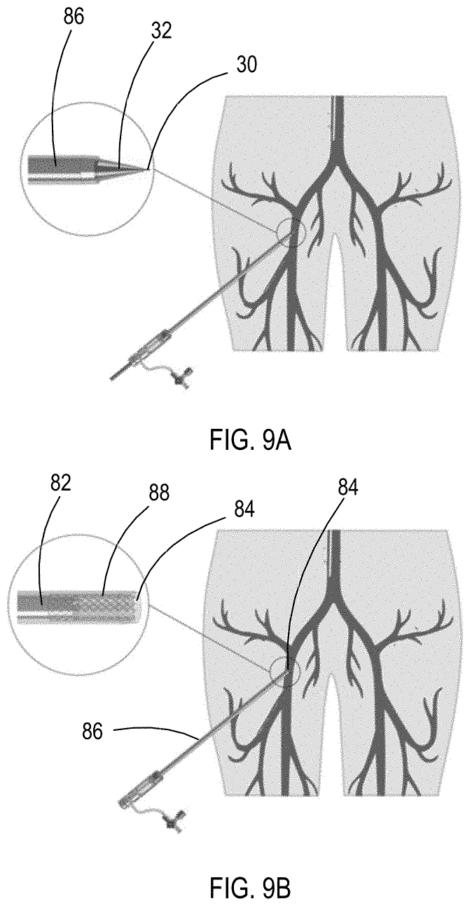

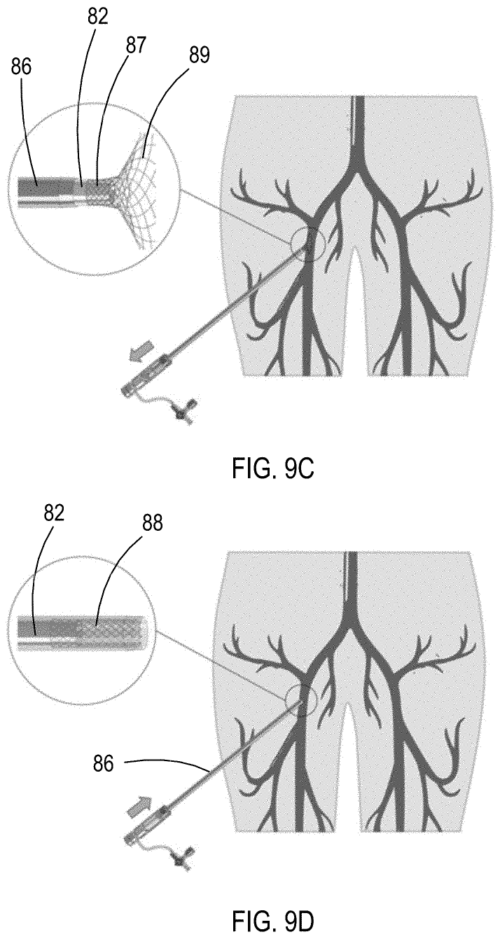

FIGS. 9D through 9D illustrate exemplary steps of using device 80 within the patient's vasculature. FIG. 9A illustrates cannula 82 having retractable sheath 86 disposed thereon in the first delivery position, and dilator 32 positioned within its lumen, advanced over guidewire 30. For example, guidewire 30 may be first delivered to the target site within the patient's vasculature. Dilator 32 may be disposed within the lumen of cannula 82, thereby providing sufficient stiffness to cannula 82 for delivery over guidewire 30 to the target site. As shown in FIG. 9B, guidewire 30 and dilator 32 may then be removed, leaving cannula 82 in the desired location within the target blood vessel, e.g., the Superficial Femoral Artery. As further shown in FIG. 9B, anchor 88 is disposed within the lumen of retractable sheath 86 in a collapsed position. In addition, outlet 84 of cannula 82 is positioned adjacent the entry site into the blood vessel to provide ample space for blood to exit outlet 84 and flow unimpeded in both antegrade and retrograde directions upon operation of device 80.

As illustrated in FIG. 9C, retractable sheath 86 may be retracted proximally relative to cannula 82, thereby causing distal expandable portion 89 of anchor 88 to be exposed beyond sheath 86 within the blood and to transition from the collapsed state to the expanded state within the blood vessel. In the expanded state, anchor 88 engages the vessel wall to anchor cannula 82 in position relative to the blood vessel, and forms a seal against cannula 82 and the entry site to the blood vessel to prevent leakage. Accordingly, during operation of device 80, e.g., via an ECMO system, blood is permitted to flow through cannula 82 and exit outlet 84 into the blood vessel and flow in both a retrograde direction toward the patient's heart and in an antegrade direction toward the patient's lower extremities, as cannula 82 does not block the blood vessel.

Upon completion of the cardiopulmonary procedure, or when diversion of blood flow to the patient's lower extremities are no longer needed, retractable sheath 86 may be moved distally relative to cannula 82, thereby transitioning distal expandable portion 89 of anchor 88 from the expanded deployed state back to the collapsed state within retractable sheath 86, as shown in FIG. 9D. Device 80 may then be removed from the patient's vasculature.

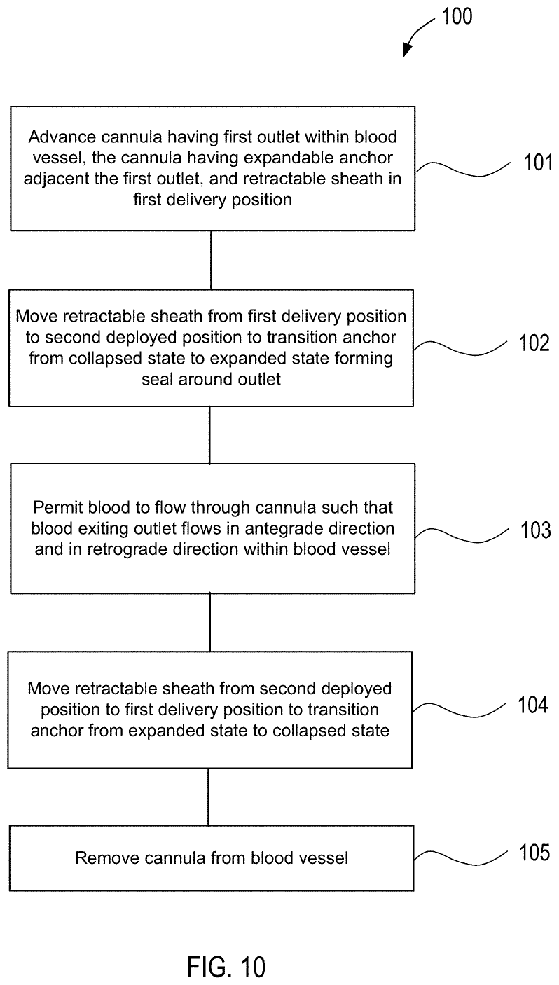

Referring now to FIG. 10, a flow chart of steps in exemplary method 100 for diverting blood flow within the patient's vasculature in accordance with the principles of the present invention is provided. At step 101, cannula 82 is advanced to the target blood vessel, e.g., the Superficial Femoral Artery, through an entry site such that outlet 84 of cannula 82 is in fluid communication with the target blood vessel, without cannula 82 blocking the flow path within the blood vessel as would be the case with a normal large sheath. Specifically, blood is permitted to flow naturally from the heart toward the patient's lower extremities when cannula 82 is in its desired position at the entry site of the target blood vessel. As described above, a guidewire may be delivered to the target site first, such that cannula 82, having an optional dilator disposed therein, may be advanced to the target site within the blood vessel over the guidewire. As will be understood by a person having ordinary skill in the art, alignment of outlet 84 with the lumen of the target blood vessel lumen may be verified using visualization methods known in the art such as fluoroscopy.

At step 102, retractable sheath 86 is moved proximally relative to cannula 82 from a first delivery position to a second deployed position, thereby causing distal expandable portion 89 of anchor 88 to be exposed beyond sheath 86 and transition from a collapsed state to an expanded state within the blood vessel. In the expanded state, distal expandable portion 89 anchors cannula 82 to the blood vessel and form a seal at the entry site to prevent blood from exiting via the entry site. At step 103, during operation of device 80, blood is permitted to flow through cannula 82 such that blood exiting outlet 84 flows into the blood vessel in a retrograde direction toward the patient's heart, and in an antegrade direction toward the patient's lower extremities. At step 104, retractable sheath 86 is moved proximally relative to cannula 82 from the second deployed position back to the first delivery position, thereby causing distal expandable portion 89 of anchor 88 to transition from the expanded state within the blood vessel to the collapsed state within retractable sheath 86. At step 105, device 80 may be removed from the patient's vasculature.

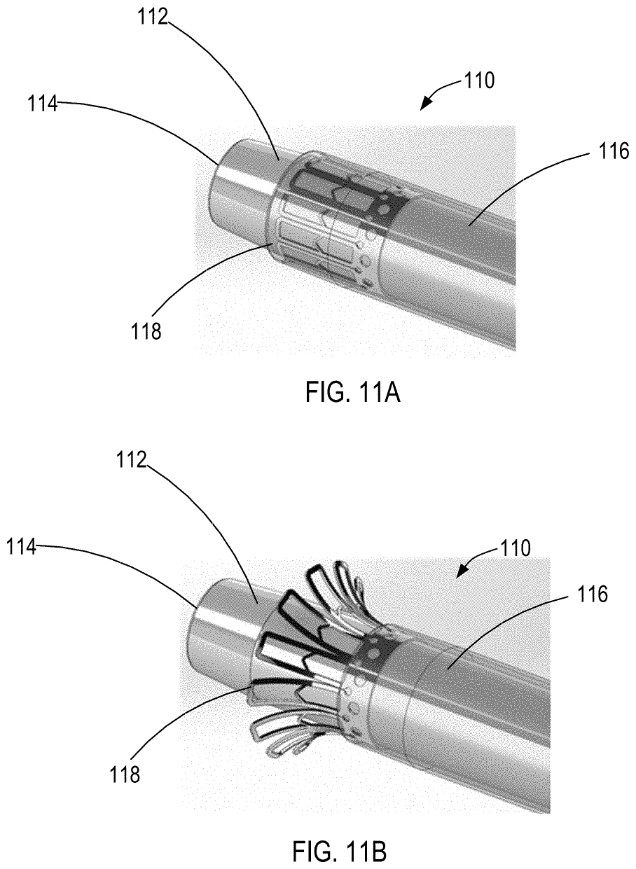

Referring now to FIGS. 11A and 11B, another exemplary device 110 for anchoring arterial cannula 112 to a blood vessel to divert blood flow within the patient's vasculature in accordance with the principles of the present invention is provided. Device 110 includes cannula 112 having outlet 114, and expandable anchor 118 coupled to the distal end thereof adjacent to outlet 114. Like anchor 88, anchor 114 has a portion that remains fixedly coupled to cannula 112 adjacent outlet 114, and a distal expandable portion which is transitionable between a collapsed delivery state and an expanded deployed state. Device 110 further includes retractable sheath 116 disposed on cannula 112. For example, in a delivery state, retractable sheath 116 is disposed over cannula 112 such that anchor 118 is collapsed within retractable sheath 116 as shown in FIG. 11A.

As illustrated in FIG. 11B, sheath 116 may be retracted to expose the distal portion of anchor 118 within the blood vessel, to thereby anchor cannula 112 to the vessel wall. In addition, the distal portion of anchor 118 may be formed from a pre-formed biocompatible metal material having a rectangular pattern such that upon expansion, the tips of the distal portion of the anchor expand radially outward away from the longitudinal axis of cannula 112. Moreover, anchor 88 may be encapsulated by a flexible membrane such that as the distal portion anchors cannula 112 to the blood vessel, anchor 118 also forms a seal around the cannula adjacent to outlet 114 and the blood vessel to prevent blood from exiting the blood vessel via the entry site.

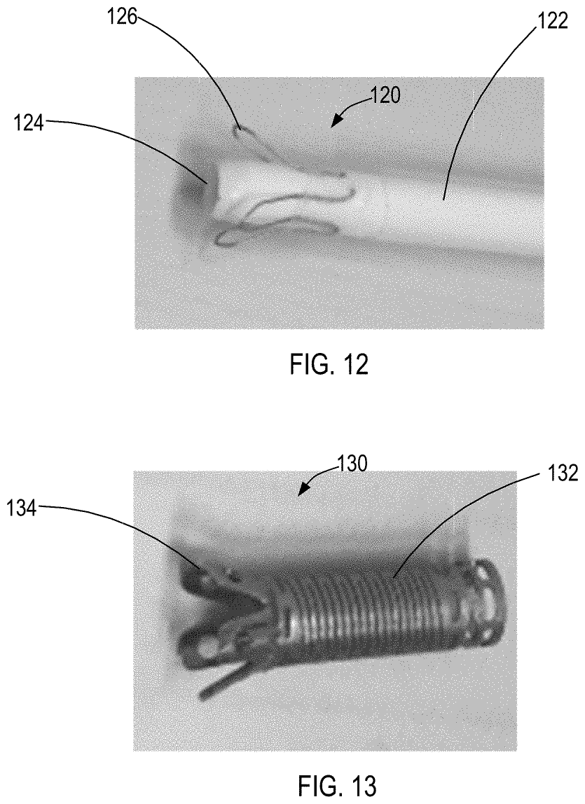

Referring now to FIGS. 12 and 13, alternative devices for anchoring an arterial cannula to a blood vessel to divert blood flow within the patient's vasculature in accordance with the principles of the present invention are provided. For example, as shown in FIG. 12, anchor 126 for anchoring cannula 122 to the blood vessel such that blood exiting outlet 124 of cannula 122 flows into the blood vessel in both antegrade and retrograde directions, may be formed of a pre-formed biocompatible wire having a petal-shaped distal portion for engaging with the vessel wall. As illustrated in FIG. 13, wherein the arterial cannula is not shown, anchor 132 for anchoring a cannula to the blood vessel such that blood exiting the cannula flows into the blood vessel in both antegrade and retrograde directions, may be formed of a pre-formed biocompatible metal having a flat petal-shaped distal portion for engaging with the vessel wall.

While various illustrative embodiments of the invention are described above, it will be apparent to one skilled in the art that various changes and modifications may be made herein without departing from the invention. It will further be appreciated that the devices and methods described herein may be utilized for diverting blood within other blood vessels within the patient's vasculature. For example, the inventive concepts herein may be used with other large catheter-based devices such as for repairing valves or other parts of the heart or vascular system, e.g., aortic or mitral valve repair or replacement or Abdominal Aorta Aneurysm stent grafts, wherein the devices have large heads but smaller shafts such that only the smaller shafts remain within the sheath during operation. The appended claims are intended to cover all such changes and modifications that fall within the true spirit and scope of the invention.

What is claimed:

1. A device for perfusing a patient's vasculature, the device comprising:
a cannula having a proximal region, a distal region having an outlet, and a lumen extending therebetween, the outlet sized and shaped to be in fluid communication with a blood vessel; and
a retractable sheath disposed on the cannula, the retractable sheath transitionable between a first delivery position and a second deployed position,
wherein, when the retractable sheath is in the second deployed position, blood flowing through the cannula is permitted to flow in an antegrade direction and a retrograde direction within the blood vessel.

2. The device of claim 1, wherein the outlet is sized and shaped to be in fluid communication with the Superficial Femoral Artery.

3. The device of claim 1, wherein the lumen of the cannula is sized and shaped to receive a dilator therethrough.

4. The device of claim 1, wherein, when the retractable sheath is in the second deployed position, blood flowing through the cannula is permitted to flow in a retrograde direction and an antegrade direction toward a lower extremity of the patient.

5. The device of claim 1, further comprising a pump coupled to the proximal region of the cannula, the pump configured to pump oxygenated blood through the lumen of the cannula.

6. The device of claim 5, wherein the pump comprises an extracorporeal membrane oxygenation (ECMO) system.

7. The device of claim 1, further comprising a pull cord coupled to the retractable sheath and configured to transition the retractable sheath between the first delivery position and the second deployed position.

8. The device of claim 1, wherein the cannula comprises a side port disposed between the proximal region and the distal region and in fluid communication with the lumen, and wherein the retractable sheath comprises a side opening sized and shaped to align with the side port when the retractable sheath is in the second deployed position, such that blood flowing through the cannula is permitted to flow in the retrograde direction via the outlet and the antegrade direction via the side port and side opening within the blood vessel.

9. The device of claim 8, further comprising a collapsible tube having a first end coupled to the cannula and in fluid communication with the side port and a second end opposite to the first end, the tube configured to transition between a collapsed state when the retractable sheath is in the first delivery position, wherein the tube is disposed between the retractable sheath and the cannula, and an expanded state when the retractable sheath is in the second deployed position, wherein the tube extends from the cannula so that the second end forms a second outlet, such that blood flowing through the cannula is permitted to flow in the retrograde direction via the outlet and the antegrade direction via the second outlet within the blood vessel.

10. The device of claim 9, wherein the tube comprises a wire mesh encapsulated by a flexible membrane.

11. The device of claim 9, wherein a surface of the side opening of the retractable sheath between an inner surface and an outer surface of the retractable sheath is sloped to facilitate extension of the collapsible tube through the side opening as the retractable sheath transitions from the first delivery position to the second deployed position.

12. The device of claim 8, further comprising one or more expandable clips coupled to the cannula adjacent to the side port, the one or more clips configured to transition between a collapsed state when the retractable sheath is in the first delivery position, wherein the one or more clips are disposed between the retractable sheath and the cannula, and an expanded state when the retractable sheath is in the second deployed position, wherein the one or more clips extend from the cannula to facilitate alignment of the side port within a blood vessel lumen, such that blood flowing through the cannula is permitted to flow in the retrograde direction via the outlet and an antegrade direction via the side port and the side opening within the blood vessel.

13. The device of claim 1, further comprising an anchor coupled to the distal region of the cannula adjacent to the outlet, the anchor having an expandable portion configured to transition between a collapsed state when the retractable sheath is in the first delivery position, wherein the expandable portion of the anchor is disposed within the retractable sheath, and an expanded state when the retractable sheath is in the second deployed position, wherein the expandable portion of the anchor extends beyond the retractable sheath to thereby anchor the cannula to a blood vessel.

14. The device of claim 13, wherein, in the expanded state, the expandable portion of the anchor forms a seal between the cannula and an entry site of the cannula into blood vessel.

15. A method for perfusing a patient's vasculature, the method comprising:
advancing a distal end of a cannula having an outlet within a blood vessel of the patient, the cannula comprising a retractable sheath disposed on the cannula, the retractable sheath transitionable between a first delivery position and a second deployed position;
moving the retractable sheath from the first delivery position to the second deployed position to thereby permit blood to flow through the cannula and into the blood vessel in an antegrade direction and a retrograde direction.

16. The method of claim 15, further comprising advancing a guidewire and a dilator within the blood vessel of the patient, wherein advancing the distal end of the cannula comprises advancing the distal end of the cannula over the guidewire and the dilator.

17. The method of claim 15, wherein the cannula further comprises a side port disposed between a proximal region and a distal region of the cannula, and wherein the retractable sheath comprises a side opening sized and shaped to align with the side port, and wherein moving the retractable sheath from the first delivery position to the second deployed position aligns the side opening and the side port, such that blood flow through the cannula exits the outlet in the retrograde direction and exits the side port and side opening in the antegrade direction within the blood vessel.

18. The method of claim 17, wherein the cannula further comprises a collapsible tube extending therefrom and in fluid communication with the side port, the tube transitionable between a collapsed state when the retractable sheath is in the first delivery position, wherein the tube is disposed between the retractable sheath and the cannula, and an expanded state when the retractable sheath is in the second deployed position, wherein the tube extends through the side opening forming a second outlet, and wherein moving the retractable sheath from the first delivery position to the second deployed position transitions the tube from the collapsed state to an expanded state, such that blood flow through the cannula exits the outlet in the retrograde direction and exits the second outlet in the antegrade direction within the blood vessel.

19. The method of claim 17, wherein the cannula further comprises one or more expandable clips extending therefrom adjacent to the side port, the one or more expandable clips transitionable between a collapsed state when the retractable sheath is in the first delivery position, wherein the one or more clips are disposed between the retractable sheath and the cannula, and an expanded state when the retractable sheath is in the second deployed position, and wherein moving the retractable sheath from the first delivery position to the second deployed position transitions the one or more expandable clips from the collapsed state to the expanded state to thereby facilitate alignment of the side port within the blood vessel, such that blood flow through the cannula exits the outlet in the retrograde direction and exits the side port and side opening in the antegrade direction within the blood vessel.

20. The method of claim 15, wherein advancing the distal end of the cannula within the blood vessel of the patient comprises advancing the distal end of the cannula through an entry site in the blood vessel such that the outlet is in fluid communication with the blood vessel without the cannula wholly blocking the blood vessel, and wherein the cannula further comprises an expandable anchor disposed on the distal end thereof, and wherein moving the retractable sheath from the first delivery position to the second deployed position transitions the expandable anchor from a collapsed state to an expanded state to anchor the cannula to the blood vessel and to form a seal between the cannula and the blood vessel at the entry site, such that blood flow through the cannula exits the outlet into the blood vessel in an antegrade direction and a retrograde direction.
